(19) United States
(12) Patent Application Publication
(10) Pub. No.: US 2009/0292354 A1
(43) Pub. Date: Nov. 26, 2009

(54) OPTIMIZED INTRAOCULAR LENS

(75) Inventors: Ivair Gontijo, Los Angeles, CA (US); Alexei Ossipov, Laguna Niguel, CA (US); Thomas R. Paul, Westlake Village, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: STAAR SURGICAL COMPANY, Monrovia, CA (US)

(21) Appl. No.: 12/124,994

(22) Filed: May 21, 2008

(51) Int. Cl. A61F 2/16; A61B 19/00

(52) U.S. Cl. 623/6.11; 128/898

(57) ABSTRACT

An optimized aspheric lens has improved optics when implanted into a patient having a curved retina. Light entering the optimized aspheric lens on-axis or at an angle to the optical axis is properly focused by the lens, reducing aberrations and producing a much smaller spot size of light on the retina. A method of selecting the optimized values for variables of the lens design, such as base radii of the front and back surface of the lens, conic constants of the front and back surfaces, and/or center thickness of the lens, among other possible parameters is provided. The method includes calculating changes in a merit function while changing the various values for the variables and selecting an optimized merit function.

### BACKGROUND OF THE INVENTION

1. Field of the Invention

The present invention relates generally to intraocular lenses (IOLS). One use for such an IOL is the replacement of a patient's crystalline lens in a cataract surgery. Another use for such an IOL is implantation into the eye of a patient needing refractive surgery.

More specifically, the present invention relates to an aspheric intraocular lens (IOL) that is optimized for the non-planar anatomy of an eye of a patient. The present invention further relates to methods for optimizing the optical features and parameters of an intraocular lens.

2. Brief Description of Related Art

Spherical and aspheric lenses are used in cataract and refractive surgeries. Spherical lenses for cataract surgery are well known in opthalmology and are available in various designs made from various materials. Recently, a number of aspheric lenses have been introduced for use to treat refractive error of an eye. For example, it is possible to design an aspheric lens to reduce the aberrations of the eye by measuring its aberrations with a wavefront sensor, measuring the corneal surface(s) of a patient's eye with a topographer, and incorporate these results into a mathematical model that is used to describe the aberrations of the eye and lens that need correction. An aspheric lens can then be designed to correct these specific aberrations, thus reducing the aberrations for this specific eye. A model eye reflecting the spherical aberration of an average cornea can also be used, so that the resulting aspheric lens can be beneficial for a wide population of patients.

Lenses may be improved by making one or both lens surfaces aspheric. The aspheric surface(s) is optimized in such a way that the spherical aberrations produced by the cornea can be largely corrected by an aspheric intraocular lens. Aspheric lenses, however, can have poor optical performance if they are not properly centered in the optical axis of the system where they are being used. The potential advantages of aspheric lenses might not be realized if they are decentered and or tilted when implanted in the eye. A decentered lens is a lens that is shifted along an axis that is generally perpendicular to the optic axis. A tilted lens is a lens that is rotated about a point where the optic axis intersects the lens. In cataract surgery there is a potential for the lens to be decentered and or tilted, either immediately after the surgery or due to small changes and contraction of the capsular bag following the surgery. It is therefore common for an intraocular lens to be shifted and or rotated in respect to the optical axis in clinical use. Previous known lenses may not focus light rays optimally on the retina if the lens is slightly tilted or shifted off center in relation to the optic axis of the eye.

Other monofocal aspheric lenses are known with extended depth of field while providing sufficient contrast for resolution of an image over a selected range of defocus distances. Multifocal aspheric lenses with a central portion having one refractive power and a peripheral portion with a different power are also known. Other lenses with diffractive elements and apodization may employ aspheric surfaces, including known designs for multifocal, accommodation, and improved depth of focus, and may also employ new materials, such as materials whose index of refraction varies throughout the material.

There are disadvantages to the prior known lenses. For example, prior lenses are typically optimized for an imaging surface that is a flat plane. This imaging surface is normally referred to as the “image plane” by lens designers and lens makers. Lenses designed to best focus light on an image plane have a disadvantage, however, when implanted in the curved eye. The image surface inside the eye, where the light should be brought to focus, is not a flat plane but can be approximated reasonably by a sphere. Another disadvantage of previous lenses, and especially so for aspheric ones, is the fact that the lenses are optimized for “on-axis” performance only. Therefore, if the lens is shifted or tilted in respect to the optical system, it will not provide an optimal performance when implanted in a patient.

Previous optimization methods are optimized to lens designs for focusing on a flat plane. Additionally, when a lens is optimized only for on-axis performance, it will focus light well only for light rays entering on-axis. An aspheric lens that is optimized only for light “on-axis” will generally have a poor calculated monochromatic modulation transfer function (MTF) versus spatial frequency for light entering at an angle, for example an angle of 5 degrees. An aspheric lens optimized for on-axis light only will produce through-focus spot sizes that are degraded for light entering at an angle as compared to spot sizes for light entering on-axis.

In previously known lenses optimized for on-axis light, a myopic shift is caused by the field curvature of the lens for light entering the lens at an angle. A bundle of rays entering the lens at an angle will come to an approximate focus before reaching the flat image plane, at a distance equal to the back focal length (BFL) from the lens. The lens field curvature therefore results in a myopic focus shift for light entering the lens at an angle.

Furthermore, because the lenses known in the art are not optimized for performance under these circumstances, the amount of myopic defocus will not match the best focus with the surface of the curved retina. Moreover, the aberrations present will produce a degraded MTF, spot size and image on the curved retina, which result in degraded visual acuity for light that is not on-axis and especially for peripheral vision. Potentially this could also cause haloes and bright spots for night vision. The spot size produced in this condition will show defocus, astigmatism and comma.

The present invention addresses the disadvantages of these prior known lenses by optimizing the configuration of a lens for various factors including a curved retina and light entering the lens off axis.

### SUMMARY OF THE INVENTION

Briefly and in general terms, the present invention provides a new and improved lens, for example in intraocular lens, having optimized parameters. The invention further includes a method for determining the optimized parameters for the intraocular lens. A method for making an optimized aspheric intraocular lens (IOL) takes the non-planar anatomy of an eye of a patient to be treated into account. In one aspect, the method of optimizing an intraocular lens considers the curvature of the retina as a factor for determining parameters of the lens. One use for such an IOL is the replacement of a patient's crystalline lens in a cataract surgery. Another use for such an IOL is implantation into the eye of a patient needing refractive surgery.

Another aspect of the present invention is an IOL that may be optimized for one or more positions and/or configurations of the lens when the lens is implanted in an eye. Yet another aspect of the present invention is a method of determining the parameters to be included in an optimized lens that is tolerant to decentration and tilt. Lenses formed according to the method of the present invention will provide better visual acuity and peripheral vision, as the benefits of an aspheric lens are preserved even when the lens is tilted, decentered or shifted.

At least one aspect of the present invention is that the anatomy of the eye is taken into account when making and using the IOL. Still another aspect of the present invention is the optimization of the parameters of an IOL, such that the ability of the IOL to focus light on a curved surface of a retina is maximized. Specifically, the curvature of the retina is considered when forming the IOL of the present invention, such that the IOL is optimized to produce the best possible image on the curved retina of the treated patient. Therefore, lenses manufactured according to the present invention will result in improved visual acuity and peripheral vision for a patient needing refractive and/or cataract surgery, while avoiding undesirable visual effects of halos, dark and bright spots.

Still another aspect of the present invention is optimizing a lens to properly focus light rays which enter the lens at an off angle to the visual axis. In other aspects of the invention various lens parameters are used to optimize the lens. For example, global lens quality considerations may be used in selecting a merit function for the method of optimizing the lens. Other merit functions may also be used, such as Strehl ratio, encircled energy, and the like to optimize the lens in other aspects of the invention. A computer program may be used to optimize the merit function while manipulating lens parameters. In one preferred aspect of the present invention, at least one surface of the IOL of the present invention is aspheric. Other surfaces may include toric, spherical, Fresnel, or other geometrical shaped surfaces. The two surfaces of a lens may not be identical.

A preferred aspect of the present invention is a method for optimizing the parameters of an intraocular lens that is adapted to be implanted into an eye of a patient. The method includes selecting at least a first lens parameter, selecting at least a second lens parameter, and selecting a merit function. An initial value is selected for the first lens parameter and an initial value is selected for the second lens parameter. The method further includes calculating an initial merit function value using the selected merit function, and varying at least one of the initial first value for the first lens parameter and the initial values for the second lens parameter a selected amount. Additional merit function values are calculated for each time the first value of the first lens parameter and the second lens parameter are varied. The optimized merit function value is determined from the calculated merit function values. The method further includes determining a preferred first value and a preferred second value that correspond to the optimized merit function value.

In yet another aspect of the present invention, an intraocular device includes an optimized lens having at least two lens parameters that are optimized using the above method.

### BRIEF DESCRIPTION OF THE DRAWINGS

These and other features, aspects and advantages of the present invention are described with reference to drawings of a preferred embodiment, which are intended to illustrate, but not to limit, the present invention.

FIG. 1 is an illustration of a human eye having a curved retina.

FIG. 2A is an exemplary intraocular lens.

FIG. 2B is a side view of the exemplary intraocular lens of FIG. 2A

FIG. 2C is an exemplary intraocular lens.

FIG. 2D is a side view of the exemplary intraocular lens of FIG. 2C.

FIG. 3 is a graph of a calculated monochromatic MTF versus spatial frequency, for a prior art lens, assuming that the lens is centered and that the light enters the lens on-axis.

FIG. 4 is a graph of a calculated MTF for a prior art aspheric lens configured for light “on-axis” only, illustrating the deterioration in MTF when light enters the lens at an angle of 5 degrees.

FIG. 5 illustrates calculated through-focus spot sizes of light on axis (top row) as compared to light off-axis (bottom row) produced by an aspheric lens optimized for on-axis light only.

FIG. 6 is an image plane of a prior art aspheric lens optimized for light on-axis, illustrating a myopic focus shift for light entering the lens at an angle.

FIG. 7 is a magnified view of the focal region for light entering the lens of FIG. 6 at an angle.

FIG. 8 is a schematic view of an example of an ISO eye model.

FIG. 9 illustrates a lens in a centered and untilted position in the ISO eye model of FIG. 8.

FIG. 10 illustrates the lens of FIG. 9 in a decentered position in the ISO eye model.

FIG. 11 illustrates the lens of FIG. 9 in a tilted position in the ISO eye model.

FIG. 12 illustrates the lens of FIG. 9 being optimized on its own, without the model cornea acromatic lens.

FIG. 13 illustrates light rays entering a lens on-axis.

FIG. 14 illustrates light rays entering a lens off-axis.

FIG. 15 is a graph of a calculated MTF for an optimized aspheric lens of the present invention, illustrating the improved MTF when light enters the lens at an angle of 5 degrees.

FIG. 16 illustrates calculated through-focus spot sizes of light on axis (top row) as compared to light off-axis (bottom row) produced by an optimized aspheric lens of the present invention.

FIG. 17 illustrates the lens field curvature of an optimized aspheric lens according to the present invention correctly matching the curvature of the retina and resulting in best focus for both light on-axis and light entering the lens at an angle.

FIG. 18 is a magnified view of the focal region for light entering the lens at an angle of FIG. 17.

FIG. 19 illustrates myopic shift of light rays entering an optimized lens at an angle.

### DETAILED DESCRIPTION OF THE PREFERRED EMBODIMENTS

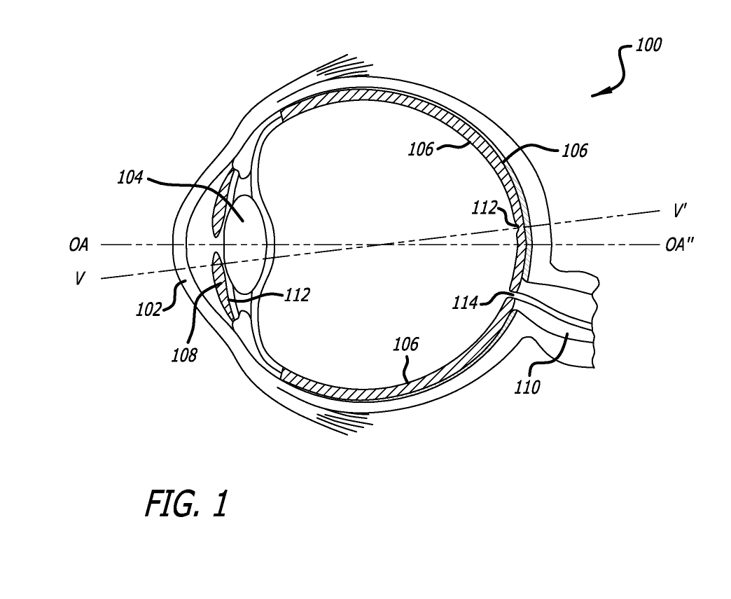

Referring first to FIG. 1 of the drawings which are provided for purposes of illustration and by way of example, the normal human eye 100 includes a curved cornea 102, a crystalline lens 104, and a curved retina 106. Light rays entering the normal human eye are refracted and focused by the cornea and the crystalline lens. Light rays enter the eye first through the cornea 102, which is the clear dome at the front of the eye. The light then progresses through the pupil 108, the circular opening in the center of the colored iris 108. Next, the light passes through the crystalline lens 104, which is located behind the iris and the pupil.

Light rays are focused through the transparent cornea 102 and crystalline lens 104 upon the retina 106. In a normal eye 100, the crystalline lens will sharply focus the light rays on the curved surface of the retina. The retina reacts to light rays falling upon it and sends impulses through the optic nerve 110 and neurons to the brain where the impulses are interpreted as images. The central point for image focus in the human retina is the fovea 112. Here a maximally focused image initiates resolution of the finest detail and direct transmission of that detail to the brain for the higher operations needed for perception. Slightly more nasally than the visual axis V-V′ is the optic axis OA-OA′ projecting closer to the optic nerve head 114. One definition of the optic axis is the longest sagittal distance between the front or vertex of the cornea and the furthest posterior part of the eyeball. It is about the optic axis that the eye is rotated by the eye muscles. The optic axis of the human eye is therefore not centered on the fovea. Furthermore, a typical human eye does not actually have a very well defined optical axis, due to many asymmetries present in the human eye.

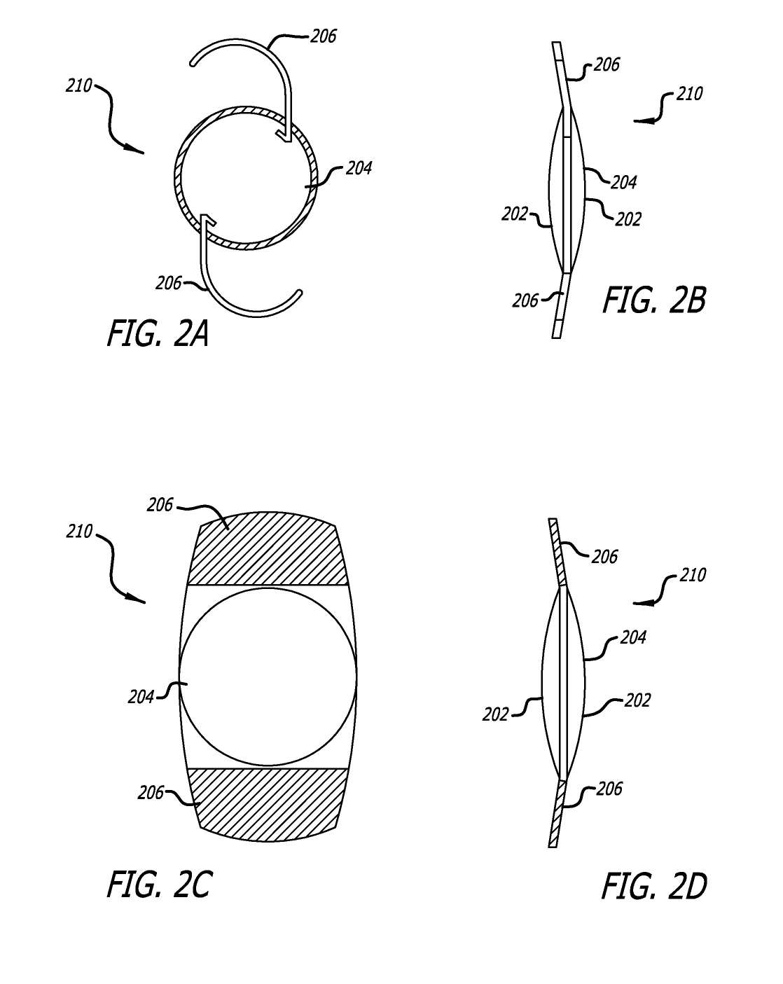

Referring now to FIGS. 2A-2C, the present invention relates to intraocular lenses (IOL) 210, which are implanted into at least one eye 100 of a patient. One use for an IOL is the replacement of a patient's crystalline lens 104 in a cataract surgery. Cataract surgery involves removing the cataractuous crystalline lens of the eye and replacing it with an IOL. Another use for an IOL is implantation into the eye of a patient needing refractive surgery. An IOL for refractive surgery may be placed without removing the patient's crystalline lens, for example, by placing the IOL in the posterior chamber 112 of the eye anterior to the crystalline lens.

Referring still to FIGS. 2A-2C, at least one embodiment of an IOL 210 includes a lens or optical zone portion 204 and may further include haptic portions 206. The optical zone portion of lens 210 focuses light rays on the retina 106. The haptics generally refer to non-focusing structures of the IOL that are added to the lens to stabilize the IOL in the eye 100 of a patient. The IOLs in these figures are by way of example and illustration, and not meant to be limiting for purposes of the present invention. In at least one embodiment, the term “IOL” may refer only to the optic portion or focusing lens of the IOL. In one embodiment of the present invention, at least one surface of the optical zone of the IOL is aspheric. Alternatively, the optical zone has a toric, spherical, Fresnel, or other geometrical shaped shape. The two surfaces of the optical zone of a lens may not be identical.

The optical zone 204 of lens 210 may be improved by imparting an aspheric geometry to one or both surfaces 202 of the optical zone 204. For convenience, hereafter it will be understood that any reference to a specific geometry of a “lens” surface is referring to the geometry of one or both surfaces of the optical zone of the IOL.

In accordance with the various embodiments of the present invention, the aspheric surface(s) of the lens is optimized in such a way that the spherical aberrations produced by the cornea 102 can be largely corrected by the aspheric intraocular lens 210. Aspheric lenses, however, can have poor optical performance if they are not properly centered in the optical axis of the system where they are being used. The potential advantages of aspheric lenses might not be realized if they are decentered and or tilted when implanted in the eye.

Perfect centration of the IOL 210 in the optical axis OA-OA′ of the human eye 100 is often difficult to achieve. Moreover, an IOL may also move within an eye after implantation, causing the lens to decenter. A decentered lens is a lens that is shifted along an axis that is generally perpendicular to the optical axis.

It is well known that the human eye may not have a very well defined optical axis, due to many anatomical asymmetries present even in the normal eye. In cataract surgery there is a potential for the lens to be decentered and/or tilted, either immediately after the surgery, or due to small changes and contraction of the capsular bag following the surgery. It is therefore common for an intraocular lens to be shifted, tilted, and/or rotated in respect to the optical axis of the eye in clinical use.

In a preferred embodiment, the present invention provides an improved intraocular lens, and a method for optimizing the performance of the lens. In one embodiment, the present invention is an optimized aspheric intraocular lens (IOL) that takes the non-planar anatomy of an eye of a patient into account when configuring the lens. In accordance with various embodiments of the present invention, the lens is configured by optimizing various parameters to maximize tolerance of the optical performance of the lens to tilt, decentration, or other factors.

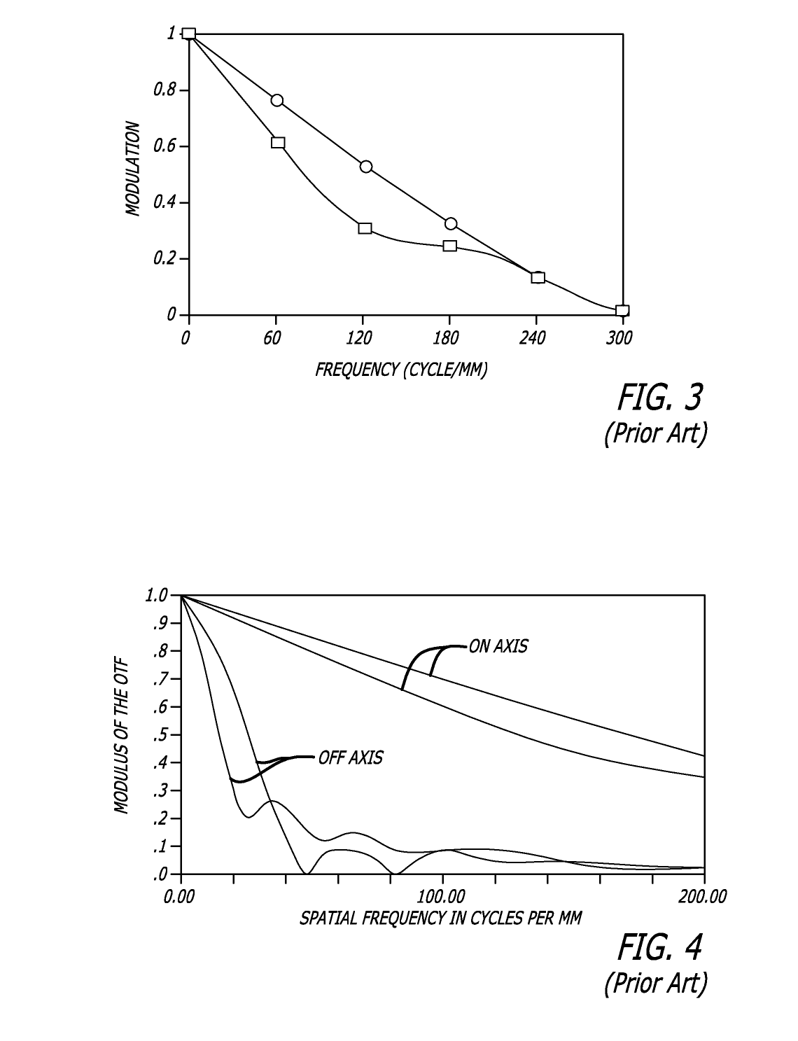

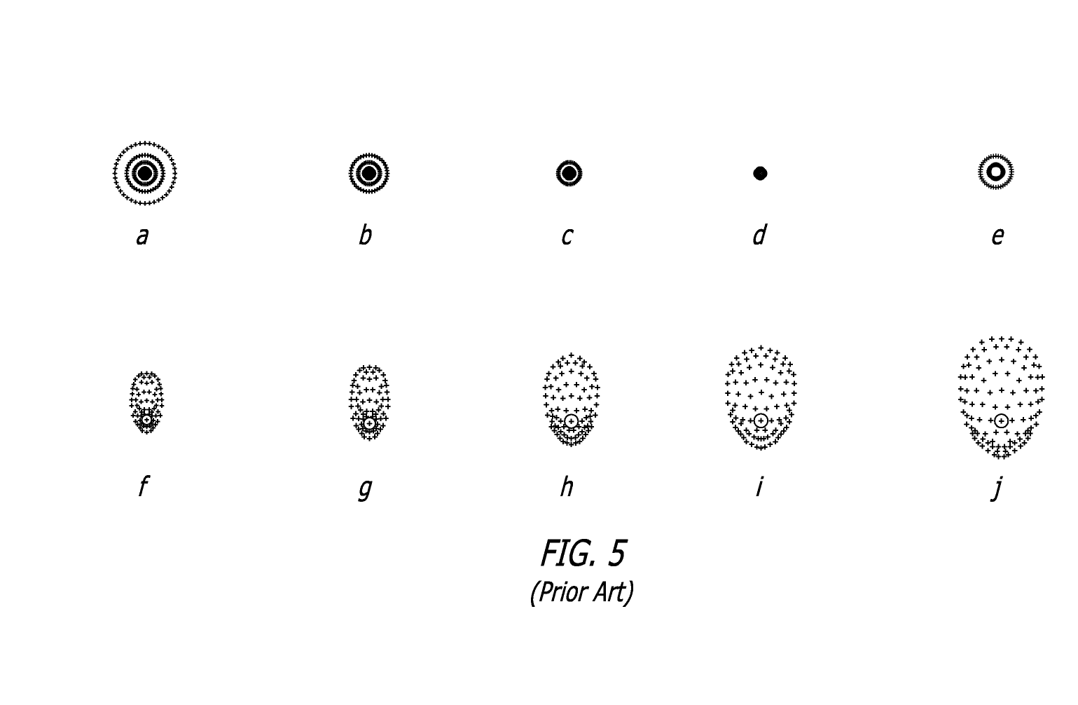

Previously known lens optimization methods and designs took into account a substantially flat image plane. When a lens is optimized only for on-axis performance, it will focus light well only for light rays entering on-axis. FIGS. 3-7 show this “prior art” situation for a 20.0 D lens. FIG. 3 is a graph of calculated monochromatic modulation transfer function (MTF) versus spatial frequency for a prior art lens assuming that the lens is centered and that the light enters the lens on-axis. As illustrated in FIG. 4, a prior art 20.0 D aspheric lens, which is optimized only for light “on-axis,” will have a poor MTF for light entering at an angle, for example, an angle of 5 degrees relative to the optic axis. Referring specifically now to FIG. 5, the spot sizes produced by a prior art 20.0 D aspheric lens are shown. The top row (spots a-e) of FIG. 5 shows the five “through focus” spot sizes for light entering the lens on-axis. The bottom row of FIG. 5 shows five “through focus” spot sizes for light entering the same prior art lens at an angle, or off-axis. These spot diagrams are known in the art as “through focus” spot diagrams because they show the light focusing properties of the lens system from a point before best focus to a point after best focus.

The first spot (a) on the top row is the spot of light formed by the prior art lens when the image plane is shifted 100 microns towards the lens (a myopic shift, or −100 micron shift). The second spot (b) in the same row represents the light focused by the lens at the image plane shifted 50 microns towards the lens. The third spot (c) represents the image produced by the lens at the best focus image plane and the next two spots (d and e) show positive focus shift (away from the lens) of +50 microns and +100 microns.

Referring now to the bottom row of spots representing the case where light is entering the prior art lens off-axis, or at an angle, it can be seen that light is being focused at a position closer to the lens that the best image plane (best focus) of the on-axis case illustrated by the spots of the top row, because the first spot (f) in the bottom row is the smallest in the bottom row. The small size of the spot (f) occurs because the light achieved the tightest focus at a point closer to the lens than the position of the first spot (−100 micron). Therefore, when this light reaches the third spot (h) in the lower row (corresponding to the best focus for light entering the lens on axis); it has accumulated a large amount of defocus. This prior art lens shows a large myopic defocus shift that is typical of lenses optimized for light on-axis only. The light entering the lens at an angle is focused a point that is uncontrolled and not-constrained by the lens design. Therefore such a lens may produce a correctly focused image for light on-axis, but a blurred image for light entering the lens at an angle.

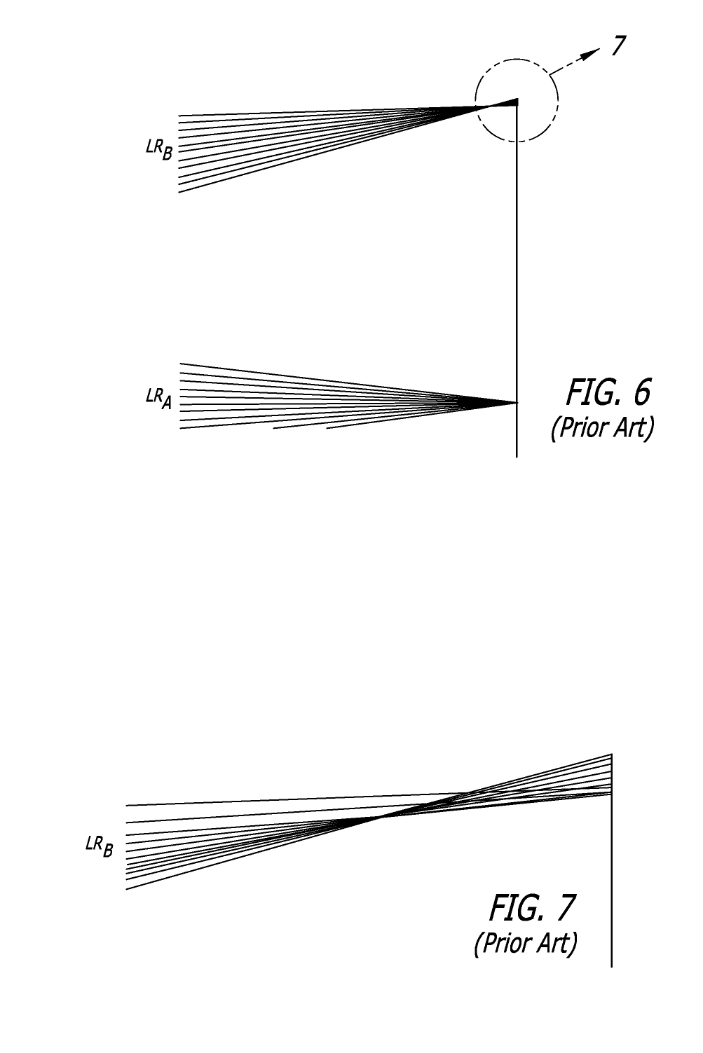

FIGS. 6 and 7 illustrate an image plane of a prior art 20.0 D aspheric lens optimized for light on-axis. As shown in FIG. 6, the light rays LRA, which are light rays that are entering the lens on axis, are generally well focused on the retinal surface. However, as shown in FIGS. 6 and 7, light rays LRB, which are light rays that are not entering the lens on axis, are generally not well focused on the curved retinal surface, thereby resulting in a myopic shift. The myopic shift is seen better in FIG. 7, which is a magnified view of angled light rays LRB poorly focused on the retinal surface.

In previously known lenses optimized for light on-axis, the myopic shift is caused by the field curvature of the lens when light enters the lens at an angle. A bundle of light rays LR entering the lens at an angle will come to an approximate focus before reaching the flat image plane, at a distance equal to the back focal length from the lens. However, because the lens is not optimized for performance under these circumstances, the amount of myopic defocus will not match the best focus with the surface of the curved retina. The lens field curvature results in a myopic focus shift for light entering the lens at an angle. The spot size produced in this condition will show defocus, astigmatism and comma. Moreover, the aberrations present will produce a degraded MTF, spot size and image on the curved retina, which result in degraded visual acuity for light that is not on-axis and especially for peripheral vision. This degradation may potentially cause haloes and bright spots for night vision.

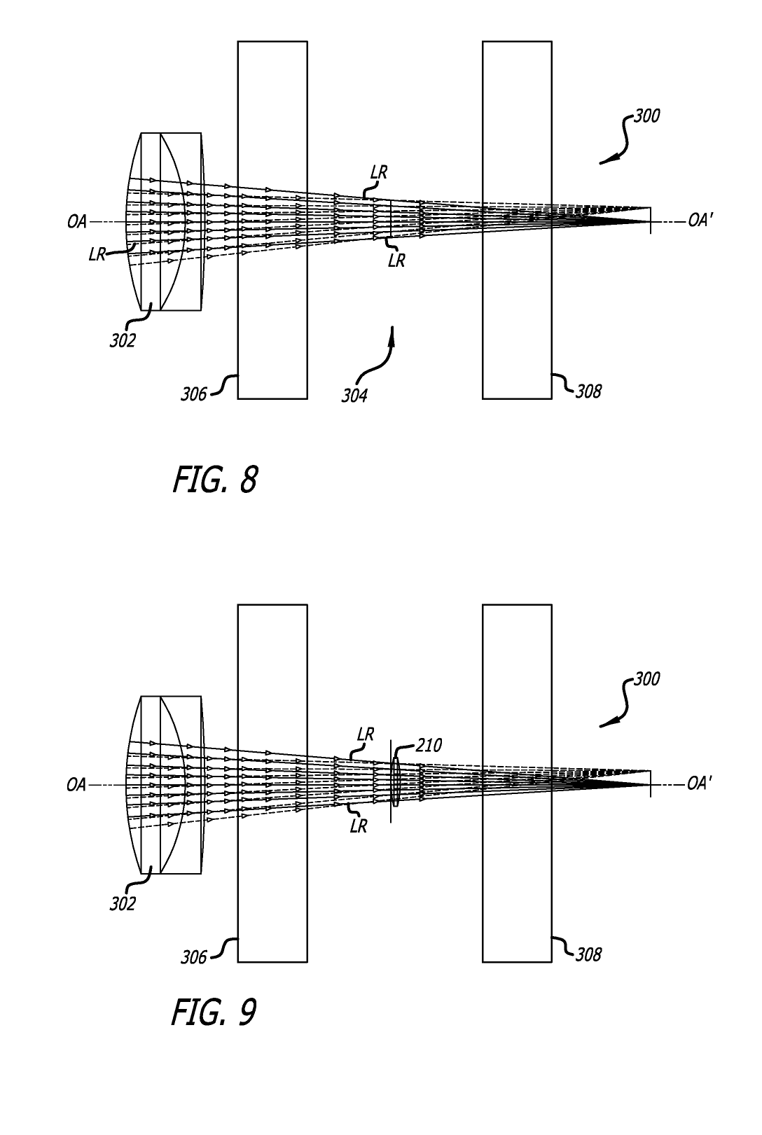

The present invention is advantageous because it provides an optimized lens 210 (FIG. 8) that correctly focuses light onto a curved retina 106 of an eye. The lens of one embodiment of the present invention is optimized both for light entering on-axis and for light entering at an angle or off-axis. Furthermore, the lens of one embodiment of the present invention is more tolerant to tilt and decentration than previous known lenses when implanted into the eye 100 of a patient. The lens of one embodiment of the present invention is optimized to produce the best focus possible even if it is tilted and/or decentered when implanted in the eye of the patient.

One embodiment of the present invention is a lens 210 having an optical portion or zone optimized for a curved retina 106 of an eye 100 of a patient (FIG. 1). The lens may have at least one aspheric surface. In other embodiments, the lens may have at least one surface that is spherical, toric, Fresnel, and/or higher order conic surface. The lens of the present invention has improved tolerance to tilt and/or decentration when implanted in an eye of a patient to be treated. The optimized lens has improved visual acuity and peripheral vision for a patient needing refractive and/or cataract surgery, while avoiding undesirable visual effects of halos, dark and bright spots.

The method of the present invention includes determining optimal lens parameters to form a lens 210 having preferred performance when implanted into an eye of a patient. The preferred performance may include better ability to focus light rays LR on a curved retina and/or the fovea of the eye, and better ability to focus light entering on-axis and off-axis. The preferred performance may also include a better ability to tolerate tilt and/or decentration of the lens. The optimized lens parameters are features of the lens composition and structure that are formed into the lens during its manufacture. The shape and intrinsic optical properties of a lens, for example, are among the parameters used to form the lens. Lens parameters may further include, for example, the dimensions of the lens, the material composition of the lens, and/or intrinsic optical properties of the lens. Still further examples of lens parameters include effective focal length (EFL), edge thickness, center thickness, optical path difference (OPD), and/or modulation transfer function. Optical path difference and modulation transfer function parameters may differ for light rays LR entering the lens at substantially different angles to the optical axis of the system. Yet other examples of lens parameters include base radius of the surfaces of the lens and/or conic constant of the surfaces of the lens. The method of the present invention may further include other lens parameters known in the art may and additional factors may be used to optimize and/or form the lens for best focusing performance on a curved retina.

Another embodiment of the present invention includes a method for determining lens parameters, which are then altered to optimize the performance of the lens. In at least one embodiment, the field curvature of the lens is optimized so that it matches the curvature of the retina 106. The lens may be optimized for one or more positions and/or configurations of the lens when the lens is implanted in an eye. In at least one other embodiment, the lens may be optimized for various angles of light entering the lens. In one embodiment, the lens is optimized at least for light entering at an angle, for example, at an angle of about 2 to 10 degrees. In at least one embodiment, the lens is optimized for light on-axis.

Referring now to FIGS. 8-12, in one further embodiment, the lens 210 is optimized in relation to the optical axis OA-OA′ of an eye model 300 or system. For simplicity, lens 210 is shown without haptics or other positioning, locating or support portions. One skilled in the art will immediately understand that haptics or other positioning portions will be used to position the lens in the eye without limiting the scope of the invention in any way.

The eye model or system may be an anatomical human eye model, or an ISO (International Organization for Standardization) eye model 300 such as the one discussed in more detail below. In at least one embodiment, an ISO eye model may be part of the lens optimization system of the present invention. Referring to FIG. 8, the ISO eye model includes a model cornea acromat lens 302. The ISO eye model further includes a vessel 304 that can hold a solution similar to the aqueous humor within the human eye. The vessel includes a first glass plate 306 connected with a second glass plate 308. The ISO eye model has very little aberrations and is close to diffraction limited. In yet another embodiment, other eye models could be used to define the optical system into which the lens is optimized. For example, different cornea models could be used. A biologically accurate eye model could also be used, representing a real cornea or an average cornea.

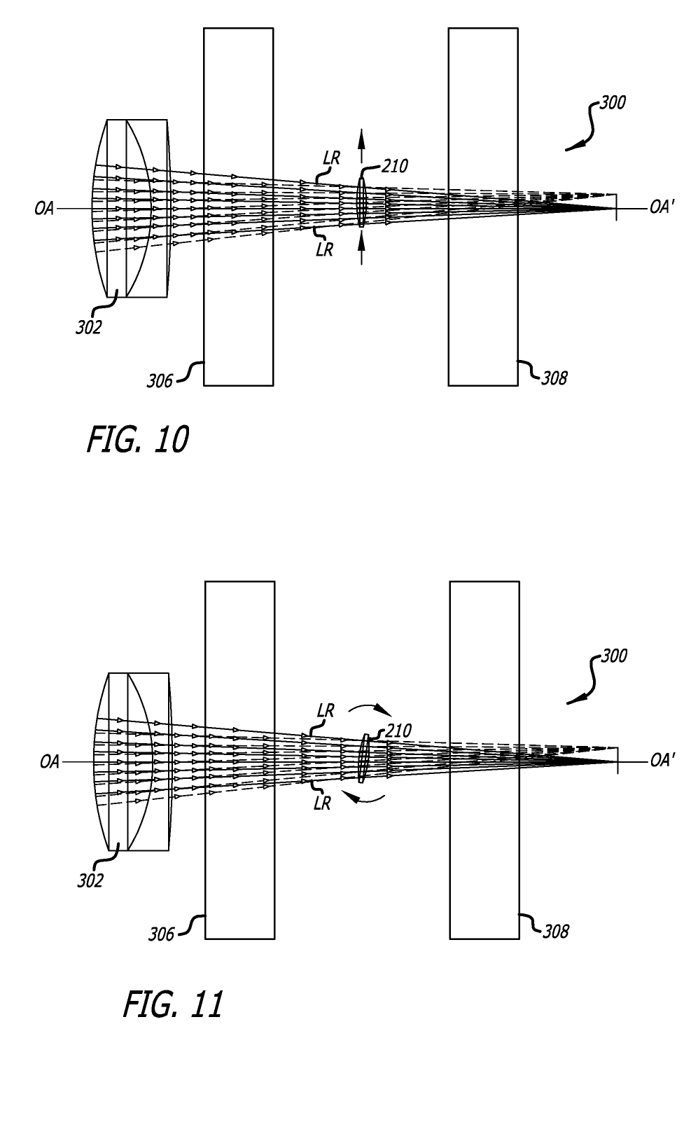

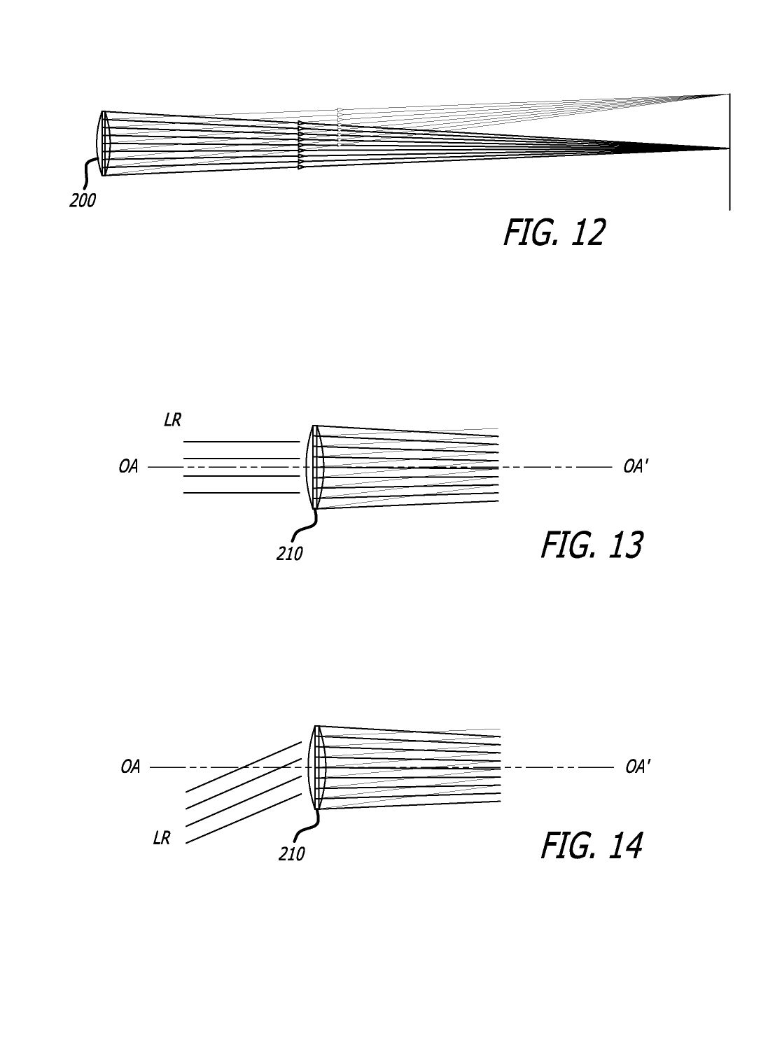

Referring to FIGS. 9-12, the lens 200 positions and/or configurations inside the ISO eye model may be changed. Referring to FIG. 9, in one embodiment, the lens is optimized while it is positioned at least aligned with the optical axis of the eye model. Referring to FIG. 10, in still another embodiment, the lens is optimized while it is at least decentered or shifted relative to the optical axis of the eye model. Referring to FIG. 11, in yet another embodiment, the lens is optimized while it is at least tilted relative to the optical axis of the eye model. Referring to FIG. 12, in a further embodiment, the lens is at least optimized on its own. The lens may be optimized on its own outside the ISO eye model and/or without using the model cornea acromat lens. In one embodiment, at least two of the optimizations are performed contemporaneously, such that the resulting optimized lens represents the best performing lens under multiple factors. In at least one embodiment, the best performing lens is selected using a selected merit function. In one embodiment, the merit function is optimized using a computer program and a computer. The merit function may include consideration of more than one factor and/or parameter when optimizing the lens. The method of the present invention may be used to produce a lens with improved tolerance to tilt and/or decentration when implanted in an eye of a patient.

Several factors affecting the performance of an implanted lens 210 may be built into the formation of the lens during its manufacturer. There are other factors that are environmental in nature. One such environmental factor is the angle at which light rays LR enter the lens. Light rays may enter the lens on-axis or various angles off-axis. At least one additional environmental factor is the wavelength of the light rays entering the lens. Additional factors affecting the performance of the lens are dependent on a patient's anatomy or the surgical technique used to implant the lens. For example, one such factor affecting the performance of a lens is the position of the lens in relation to the optical axis OA-OA′ of the system in which it is implanted. The lens may be positioned centered or decentered various amounts. The lens may also be positioned tilted or un-tilted to various degrees.

There are parameters of the lens 210 itself that may be designed into the formation of the lens during manufacturing, for example, shapes and dimensions of the lens and/or the material composition of the lens. Other lens parameters known in the art may also affect the performance of a lens. One embodiment of the present invention includes a method for determining the best lens parameters, such that the lens will perform optimally under a variety of likely environmental and anatomical implanted conditions. At least one other embodiment includes an optimized lens that is formed by using the method of the present invention.

Referring again to FIGS. 9-12, one embodiment of the present invention includes optimization of the lens for best focusing performance under typical clinical conditions, such as a curved retina and/or tilt or shift of an implanted lens in the eye. The optimized lens performs well in a human eye, which has a curved retina, and is tolerant to tilt and shift of the lens when implanted in the eye. In one embodiment, optimization of the lens is performed in four different positions or conditions. The lens may however be optimized in fewer than four positions or more than four positions. Optimization in the four positions is preferably performed contemporaneously. Referring specifically to FIG. 9, in a first position the lens is placed on-axis in the eye model. Referring specifically to FIG. 10, in a second position the lens is shifted (decentered) from the optical axis of the eye model. The lens may be decentered by various amounts. In one embodiment, for example, the lens may be decentered by a small amount, for example 0.6 mm. In a third position, as illustrated specifically in FIG. 11, the lens is tilted in relation to the optical axis of the eye model. The lens may be tilted by various degrees. In one embodiment, for example, the lens is shifted by a small angle, for example 2 to 10 degrees. In a fourth position, as illustrated specifically in FIG. 12, the lens is optimized outside of the ISO eye model.

In another embodiment, a fewer number of positions may be used during the optimization. For example, not all positions described herein need to be used together. Furthermore, in yet another embodiment, combinations of positions and/or other factors may be used during the optimization. For example, in one embodiment a lens may be optimized for best performance with the lens decentered, and tilted inside the eye model with light on-axis. In another embodiment a lens may be optimized for best performance with the lens decentered, and tilted inside the eye model with light off-axis. The positions and other factors described herein are by example and not meant to be limiting. Furthermore, any combination of light on-axis/off-axis and lens on-axis/off-axis, such as tilted or decentered, may be used in conjunction with the above positions for the optimization.

One factor that may be varied during the method of optimizing the lens 210 is the entrance angle of light rays (LR) entering the lens in relation to the optic axis OA-OA′. The entrance angle is commonly referred to as the field angle. In one embodiment, the field angle may be varied from zero degrees to another value, thereby creating additional configurations for the lens. In at least one embodiment, light rays LR enter the lens on-axis, as illustrated in FIG. 13. A standard method of optimizing an intra-ocular lens as known in the art. In another embodiment, light rays LR enter the lens at an angle to the optic axis OA-OA′, as illustrated in FIG. 14. For example, in one embodiment light rays may enter the lens at an angle of 2.5 degrees relative to the optic axis. In another embodiment light rays may enter the lens at an angle of 5.0 degrees relative to the optic axis. In still one other embodiment light rays may enter the lens at an angle of 10.0 degrees relative to the optic axis. Varying the entrance angle of the light is advantageous for simulating light reaching the retina 106 (FIG. 1) at different angles, thus simulating peripheral vision. For example, light entering at an angle of 2.5 degrees, may correspond to a spot about 1.25 mm away from the center of a spot formed by light on-axis. This retinal location corresponds roughly to the edge of the fovea 112, which is the area of highest visual acuity. Additional factors may be included to represent variations in the optical system, for example the shape of the retina.

In one preferred embodiment of the present invention, the optimization method includes the calculation of an initial value of a selected merit function (MF), using selected initial values for lens parameters and/or other factors. A merit function, also known as a figure-of-merit function, is a function that measures the agreement between data and the fitting model for a particular choice of the parameters. By convention, the merit function is generally small when the agreement is good. In a process known as regression, parameters are adjusted based on the value of the merit function until a smallest value is obtained, thus producing a best-fit with the corresponding parameter values giving the smallest merit function value. These will be the chosen best-fit parameters used to form the optimized lens. The merit function for the lens optimization expresses how well the optics of a lens perform. In one embodiment, the smaller the value of the merit function, the better optimized is the lens. In another embodiment, a merit function is constructed that increases as the lens performs better, such that a maximization of the merit function is sought when optimizing the lens performance.

Variations in the values of the lens parameters will result in a change in value of the calculated merit function. A lens parameter may also include an optical feature of the lens when the lens is in a selected position. In at least one embodiment of the present invention, the value of one or more parameters of the lens is changed, resulting in a newly configured lens, and the merit function is recalculated for the newly configured lens. In one embodiment, additional parameters may be added by taking into consideration optical performance in one or more of the positions discussed above. In at least one further embodiment, the lens position in the optical system is varied and the merit function is recalculated. One or more factors may be chosen to be contemporaneously varied when optimizing the parameters of the lens.

If the recalculated merit function is a more desirable value, the newly configured lens is better and is kept as a candidate for an optimized lens. For example, using a merit function constructed such that decreases in the MF indicate a better performing lens, if the recalculated MF value for the newly configured lens is smaller than the previous one, the new lens parameters are better and the lens is kept as a candidate for the optimized lens. This cycle of changes in the value of the factors and MF recalculations is repeated until the merit function no longer changes for small changes in the lens parameters. At this point the lens is optimized.

In at least one embodiment, the method of lens optimization of the present invention includes use of a computer program and a computer. A computer program may calculate the merit function for combinations of two or more factors. Furthermore, the computer program can calculate the merit function for a large number of lens candidates having various combinations of parameters, and various values for the parameters. Using the method of the present invention, the computer program may be used to find the lens candidate that optimizes the merit function value. For example, in one embodiment thousands or millions of lens designs can be tried to identify the optimized lens design.

In one embodiment of the present invention, a merit function may be constructed of the form:

$${MF}^{2} = \frac{{\sum }_{i = 1}^{N} {{w}_{i} ( {x}_{i} - {T}_{i} )}^{2}}{{\sum }_{i = 1}^{N} {w}_{i}}$$

### Where:

1) Tᵢ is the target value of an i-th optimization parameter, representing the optimum value of the i-th parameter;

2) xᵢ is the present value of the i-th optimization parameter;

3) wᵢ is a weight that multiplies the difference between the present and the optimum value of parameter i, thereby increasing or decreasing the importance of an optimization parameter compared to the others; and

4) N is the total number of parameters comprising the merit function.

Thus, the merit function MF is the sum of the squared differences between the target values and the present values for the candidate lens.

In various embodiments of the present invention, the parameters that are included in the optimization for a lens properly centered on the optical axis OA-OA′ of the eye model may be chosen from, for example, effective focal length (EFL), edge thickness, center thickness, optical path difference (OPD) for light entering the lens on-axis, and/or modulation transfer function for light on-axis. The effective focal length of a lens may be calculated from the optical power (P) of the lens using the formula:

EFL=P/1000 mm.

For example, in one embodiment of the method of the present invention a lens may be optimized using the following optimization parameters:
T1=Effective Focal Length (EFL)=50 mm, where the lens has an optical power of P=20.0 D;
T2=Edge Thickness=0.33 mm;
T3=Center Thickness<1.2 mm;
T4=Optical Path Difference (OPD)=0.0 for light entering the lens on-axis;
T5=Modulation Transfer Function for light on-axis, at 100 LP/mm>0.6; and
TN=Nth optimization factor.

Additional OPD and MTF parameters may be included for additional positions and/or configurations of the lens. In one preferred embodiment, at least one additional OPD and at least one additional MTF value is included, to optimize the lens when it is shifted (decentered) or tilted from the optical axis OA-OA′. The ISO eye model 300 may be used to confirm optimization, for example, when the lens is shifted (decentered) from the optical axis and/or tilted. Furthermore, in at least one other embodiment additional optimization parameters may include optical path differences (OPD) for light entering the lens at an angle, and/or modulation transfer function for light entering the lens at an angle, such that at least seven optimization parameters are used.

In one additional preferred embodiment, at least eleven optimization parameters are needed to optimize the lens in three different configurations: on-axis in the eye model, shifted in the eye model, and tilted in the eye model. More or fewer optimization parameters may be used depending on the needs of the lens designer without departing from the scope of the invention. For example, additional optimization parameters may be added for a lens shifted by 0.6 mm and/or a lens tilted by 2.5 degrees. In at least one embodiment, all weights wᵢ are set equal to 1.0. In still another embodiment, at least a curved retina surface may be used with various positions and/or configurations and/or parameters. In yet another embodiment, at least a flat image plane may be used with various positions and/or configurations and/or parameters. In a further embodiment, other and/or varying amounts of tilt or decentration could be used during the optimization method.

In still another embodiment, a constraint on the shape of the entire MTF curve may be imposed, instead of a constraint at one point of the curve only. In one embodiment, the MTF is required to be higher than 0.6 at 100 line pairs/mm. In another embodiment, a different spatial frequency for the required MTF may be chosen, such as 50 or 150 line pairs/mm.

In at least one other embodiment, a computer program is configured to calculate changes in merit function associated with variable lens parameters. In one embodiment, the parameters used to optimize the lens include base radius of the front surface of the lens (R_{f}), conic constant of the front surface (C_{f}), base Radius of the back surface of the lens (R_{b}), conic constant of the back surface of the lens (C_{b}), and/or center thickness of the lens (t_{c}). Other parameters may be included, such as the refractive index of the lens material. Alternatively, a parameter variable may be set to a constant. For example, if R_{f} is equal to R_{b}, then the optimized lens surface will be equi-convex. The optimized lens surfaces 202 (FIGS. 2B and 2D) may, however, be spherical, toric, Fresnel, or higher order conic surfaces.

In at least one preferred embodiment of the present invention, the radius curvature of the imaging surface is set to 13 mm, which represents the average radius of curvature of the retina in human adults. Therefore, at least one important advantage of the present invention over prior known IOL technology is that optimization of the a lens to inserted in an eye takes into account the curve of the human retina. In yet other embodiments, other shapes for the retina could be used, for example spherical retinas with a radius of curvature different than 13 mm. Furthermore, other geometrical shapes than spherical may be used, such as ellipsoids, paraboloids, hyperboloids or prolate or oblate versions of these surfaces. Irregular surfaces, not definable by a single equation, could be used as well.

In one embodiment of the present invention, the method of the present invention is embodied in a computer programmed to carry out the calculations and comparisons required to determine the merit function of various lens designs under various conditions, that is, with various parameters as has been discussed. The program is given a set of starting values for selected lens parameter variables and a starting merit function value (MFstart) is calculated. The optimization proceeds with numerous, for example thousands, of lens design candidates being generated by small changes in the parameter values, with the MF being recalculated for each design iteration. An optimized set of lens parameters, with a best possible merit function value (MFfinal) is thereby determined. This optimized set of lens parameters represents the best lens that can be formed for an intended use. In another embodiment, the lens is optimized to focus light on a curved retina and to have the best performance either on-axis, or when it is de-centered or tilted from the optical axis.

In still other embodiments, other merit functions that express lens quality may be used to optimize lens parameters. Examples of other merit functions that may be used are the encircled energy; the point spread function, the Strehl ratio, and/or the optical transfer function (OTF). In general, any merit function expression that describes lens quality could be used to construct a merit function that is then optimized. In yet other embodiments, parameters that describe lens problems or lens poor performance could be used to construct a merit function which is then optimized for light on-axis and off-axis and/or for different lens configurations (tilted, decentered, etc). Examples of such parameters include “the wavefront error”, “Zernike polynomials”, “Seidel aberrations”, “ray aberrations”, and the like.

### EXAMPLE

In one example, starting values are selected for five lens parameters. Starting values for R_{f} and R_{b} are both set equal to 20.0 mm, starting values for C_{f} and C_{b} are both set equal to −5.0, starting value for t_{c} is set equal to 1.0 mm, and all weights (wᵢ) are set equal to 1. The starting merit function value (MFstart) was calculated and found to be 4.259. The design was then optimized by making small changes to the parameters and recalculating the MF. This process is continued until the lowest possible MF is found. The optimized result, with the lowest possible merit function value (MFfinal) of 0.05900 was found when the parameters had values for R_{f} and R_{b} equal to 10.536 mm, C_{f} and C_{b} equal to −0.960, and t_{c} equal to 1.186 mm. This set of lens radii of curvature, conic constants and center thickness, together with the lens refractive index represents the best lens that can be configured which will satisfy all the conditions in the merit function for MTF, focal length, edge and center thickness. The optimized lens will focus light on a curved retina and will have the best performance either on-axis, or when it is de-centered or tilted from the ISO standard optical axis OA-OA′.

In another embodiment, optimizations are run for a single wavelength of light, for example 546 nm, which corresponds to green light and is the wavelength recommended in the ISO standard. In yet other embodiments, other wavelengths of light can be chosen. The optimizations may include at least two wavelengths spanning the visible (or infrared or UV) spectrum.

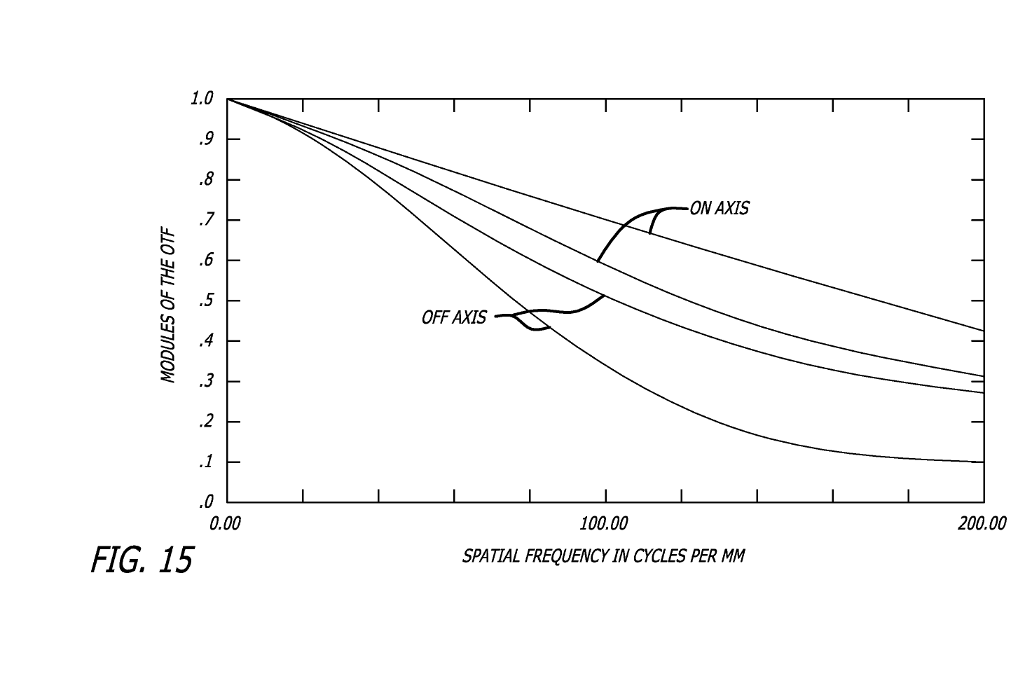

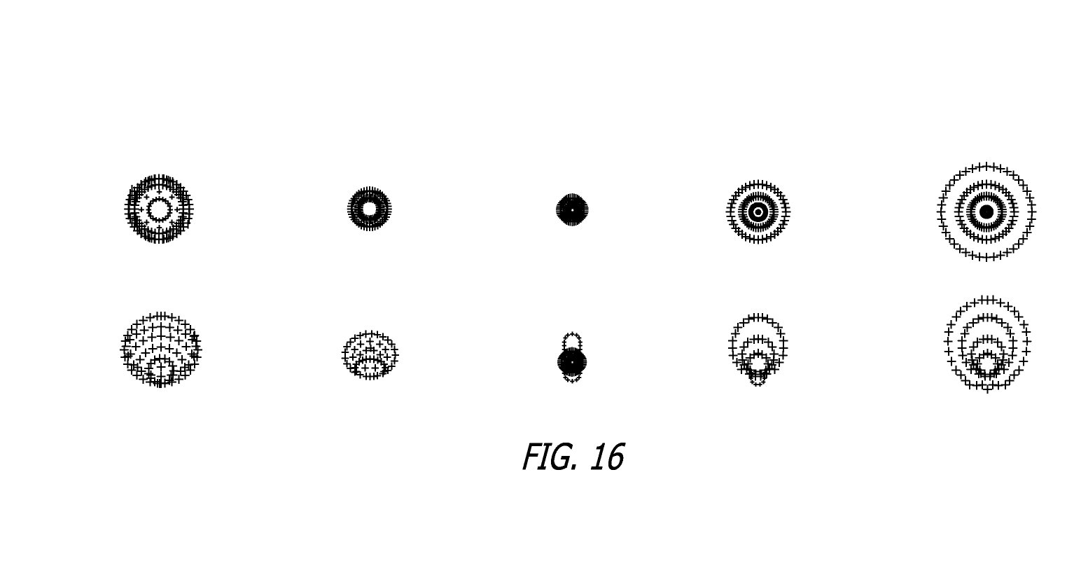

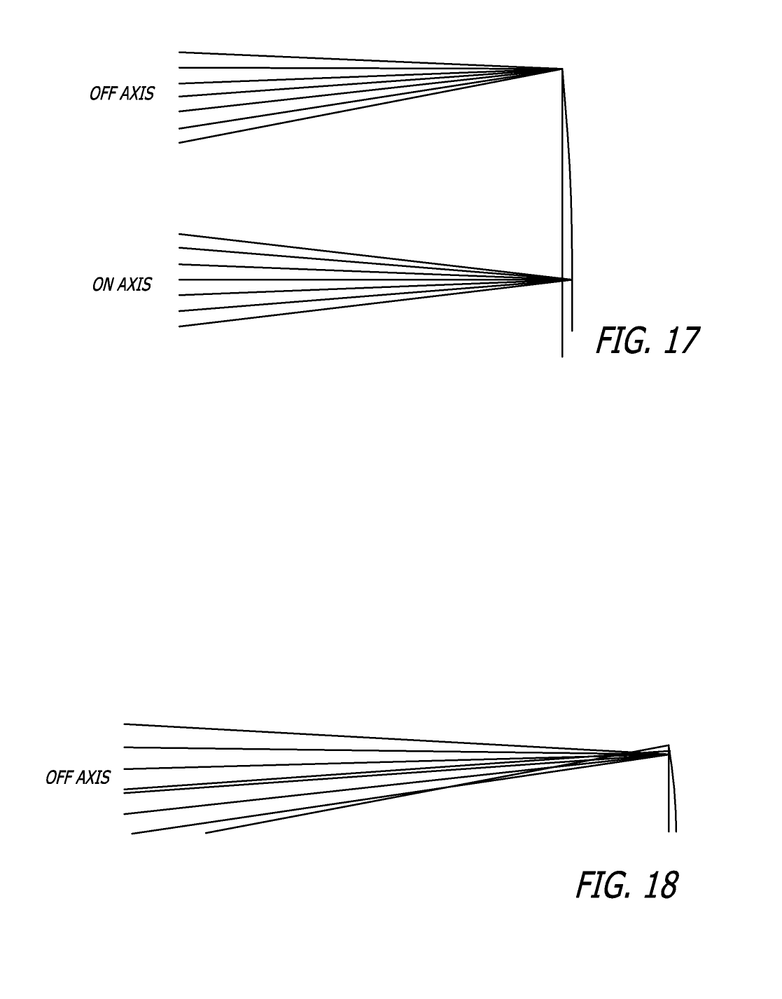

Referring now to FIGS. 15-18, the improved lens 210 of the present invention has better performance for light on-axis and light entering the improved lens at an angle. For example, referring specifically now to FIG. 15, the calculated MTF of an embodiment including a 20.0 D aspheric lens after optimization according to the method of the present invention has significant improvement in the MTFs for light entering the lens at an angle, as compared to the prior art plot of FIG. 3. The image surface of, for example, a 20.0 D aspheric lens after optimization according to the present invention is illustrated in FIGS. 16-18. Comparing the spot diagrams a lens optimized in accordance with the present invention in FIG. 16 to the spot diagrams of a prior art lens illustrated in FIG. 5 clearly shows the improvement in lens performance, particularly for the light entering the lens off-axis, when the lens is optimized. Similarly, the performance of the optimized lens illustrated in FIGS. 17 and 18 can be compared with the performance of the prior art lens depicted in FIGS. 6 and 7, clearly showing that the optimized lens is superior to the prior art lens, especially when light enters the lens at an angle.

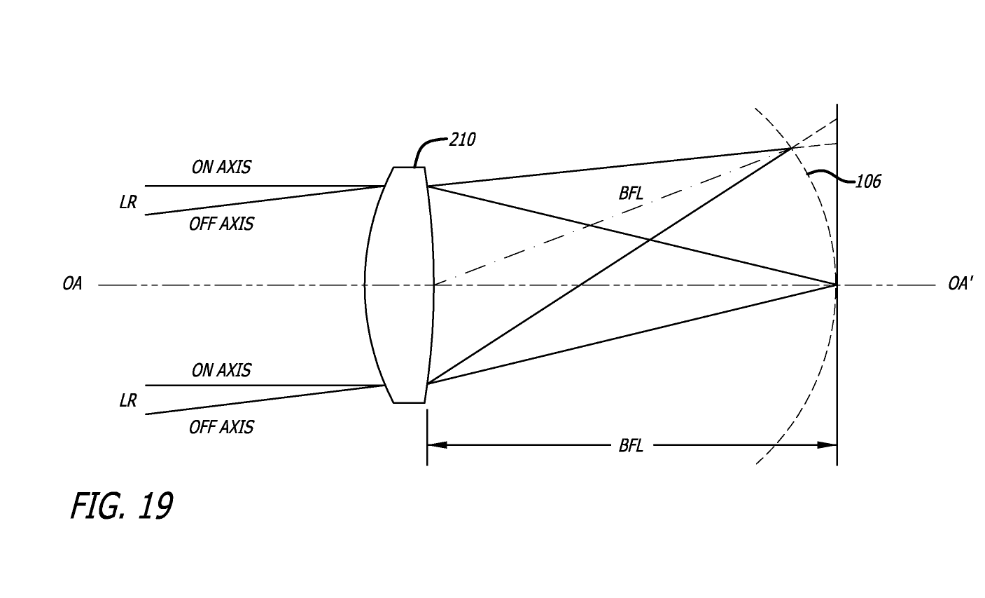

Referring also now to FIG. 19, the lens field curvature matches correctly the curvature of the retina 106 (FIG. 1), resulting in best focus for both light on-axis and light entering the lens at an angle. The light entering the optimized aspheric lens at an angle to the optical axis is properly focused in this case, reducing aberrations and producing a much smaller spot size.

The invention may be embodied in other forms without departure from the spirit and essential characteristics thereof. The embodiments described therefore are to be considered in all respects as illustrative and not restrictive. Although the present invention has been described in terms of certain preferred embodiments, other embodiments that are apparent to those of ordinary skill in the art are also within the scope of the invention. Accordingly, the scope of the invention is intended to be defined only by reference to the appended claims.

We claim:

1. A method for optimizing the parameters of an intraocular lens that is adapted to be implanted into an eye of a patient, comprising:
selecting a first lens parameter;
selecting a second lens parameter;
selecting a merit function;
selecting an initial value for the first lens parameter;
selecting an initial value for the second lens parameter;
calculating an initial merit function value using the selected merit function;
varying at least one of the initial first value for the first lens parameter and the initial value for the second lens parameter a selected amount;
calculating additional merit function values for each time the first value of the first lens parameter and the first value of the second lens parameter are varied;
determining the optimized merit function value from the calculated merit function values; and
determining a preferred value of the first lens parameter and a preferred value of the second lens parameter that correspond to the optimized merit function value.

2. The method of claim 1, further including manufacturing a lens having parameters including at least a first lens parameter having the preferred first value, and a second lens parameter having the preferred second value.

3. The method of claim 1, wherein the first parameter is selected from the group of consisting of a base radius of a lens surface, a conic constant of a lens surface, a center thickness of a lens, an effective focal length of a lens and an edge thickness of a lens.

4. The method of claim 1, wherein the first parameter represents light on axis.

5. The method of claim 1, wherein the first parameter represents light off axis.

6. The method of claim 1, wherein the first parameter represents a centered lens.

7. The method of claim 1, wherein the first parameter represents a decentered lens.

8. The method of claim 1, wherein the first parameter is an optical path difference.

9. The method of claim 8, wherein the optical path difference is for light on axis.

10. The method of claim 8, wherein the optical path difference is for light off axis.

11. The method of claim 1, wherein the first parameter is a modulation transfer function.

12. The method of claim 11, wherein the modulation transfer function is for light on axis.

13. The method of claim 11, wherein the modulation transfer function is for light off axis.

14. An intraocular device, comprising an optimized lens having at least two optimized lens parameters, wherein at least one of the optimized lens parameters is determined by the method of claim 1.

15. The intraocular device of claim 14, wherein the optimized lens includes at least one aspheric surface.

16. The intraocular device of claim 14, wherein the optimized lens includes at least surface selected from the group consisting of a spherical surface, a toric surface, a Fresnel surface and a higher order conic surface.

17. The intraocular device of claim 14, wherein the lens is optimized for a curved retina.

18. The intraocular device of claim 14, wherein the lens is optimized at least for light on axis.

19. The intraocular device of claim 14, wherein the lens is optimized at least for light off axis.

20. The intraocular device of claim 14, wherein the lens is optimized for at least one wavelength of light.

21. The intraocular device of claim 14, wherein the lens is optimized for at least two positions.
